(19) United States
(12) Patent Application Publication
(10) Pub. No.: US 2008/0208340 A1
(43) Pub. Date: Aug. 28, 2008

(54) Synthetic bone graft

(76) Inventors: Daniel Boyd, County Cork (IE); Mark Robert Towler, County Clare (IE)

Correspondence Address: JACOBSON HOLMAN;PROFESSIONAL LIMITED LIABILITY COMPANY, 400 SEVENTH STREET, N.W., WASHINGTON, DC, 20004, US

(21) Appl. No.: 12/071,756

(22) Filed: Feb. 26, 2008

Related U.S. Application Data
(60) Provisional application No. 60/903,316, filed on Feb. 26, 2007.

(51) Int. Cl. A61F 2/28; A61Q 11/00; A61K 8/27; A61K 8/18; C03C 3/00; C03C 3/04; A61K 33/30; A61P 19/10; A61P 19/08

(52) U.S. Cl. 623/16.11; 424/49; 501/11; 501/53; 424/643

(57) ABSTRACT

A particulate glass for a synthetic bone (including dental) graft includes ZnO, SrO, and may include NaO. The glass promotes cellular metabolism, and upon implantation in living bone tissue induces bone growth at their surface. The ZnO and SrO respectively degrade to provide Zn²⁺ and Sr²⁺ ions respectively. The ions released by the glass provide anti-bacterial effects; improved bone formation in place of diseased tissue; inhibition of bone resorption; and radiopacity. There is excellent synergy between the SrO, ZnO, and NaO. The Sr²⁺ ions have better bone formation effects than the Zn²⁺ ions, but an anti-bacterial effect which is not as good. Choice of relative proportions of ZnO and SrO combined with the choice of NaO concentration to set the resorption rate allow optimisation. NaO there is control of the degradation rate of the graft; a feature which is advantageous in tailoring the grafts to specific patients and applications. Additionally the sodium (Na) in the glass imparts water solubility, allowing glasses to degrade to their ionic components.

The invention relates to synthetic bone grafts.

Currently available bone graft materials have some limitations, particularly for patients with metabolic bone diseases such as osteoporosis. A common problem is that the graft is reabsorbed within a time period (some months) and replaced by diseased tissue similar to that which initially caused fracture.

The invention is directed towards providing an improved bone graft material.

### STATEMENTS OF INVENTION

According to the invention, there is provided a synthetic bone graft glass having a composition including SrO and ZnO.

In one embodiment, the glass comprises SiO₂, ZnO, CaO, and SrO.

In one embodiment, the glass further comprises NaO.

The concentration of ZnO may be in the range of 0.05 to 0.50 mole fraction, and preferably is in the range of 0.10 to 0.32 mole fraction.

The concentration of SrO may be in the range of 0.05 to 0.50 mole fraction, and is preferably in the range of 0.14 to 0.40 mole fraction.

The concentration of NaO may be in the range of 0.05 to 0.5 mole fraction, and is preferably in the range of 0.1 to 0.3 mole fraction.

In one embodiment, the glass structure is Q₂ or less, having network connectivity of 2 or less, and the network connectivity is in one example 1.

In one embodiment, the glass is in particulate form, and the particle size is preferably in the range of 350 μm to 950 μm.

In another embodiment, the glass is in the form of a load-bearing body.

The invention also provides a synthetic bone graft comprising any glass as defined above. The graft may be a void-filling graft, or a cranio-maxillo facial graft, for example.

The invention also provides a toothpaste comprising any glass as defined above.

In a further aspect, the invention provides a method of synthesising any glass defined above, in which SrO and ZnO concentrations are chosen to optimise target bone formation and anti-bacterial activities, the SrO component providing better bone formation properties and the ZnO component providing better anti-bacterial properties.

The method may comprise the further step of forming the glass into a load-bearing body.

### DETAILED DESCRIPTION OF THE INVENTION

The invention will be more clearly understood from the following description of some embodiments thereof, given by way of example only with reference to the accompanying drawings in which: —

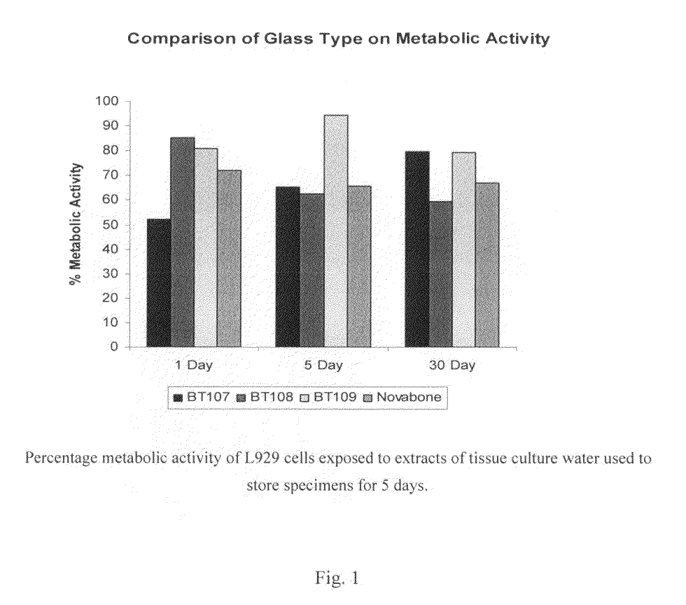

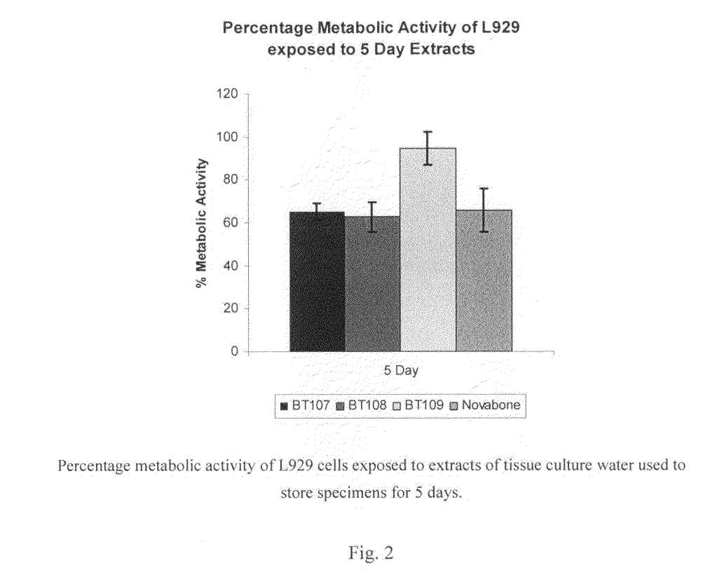

FIGS. 1 and 2 are plots indicating metabolic activity for a range of glass types;

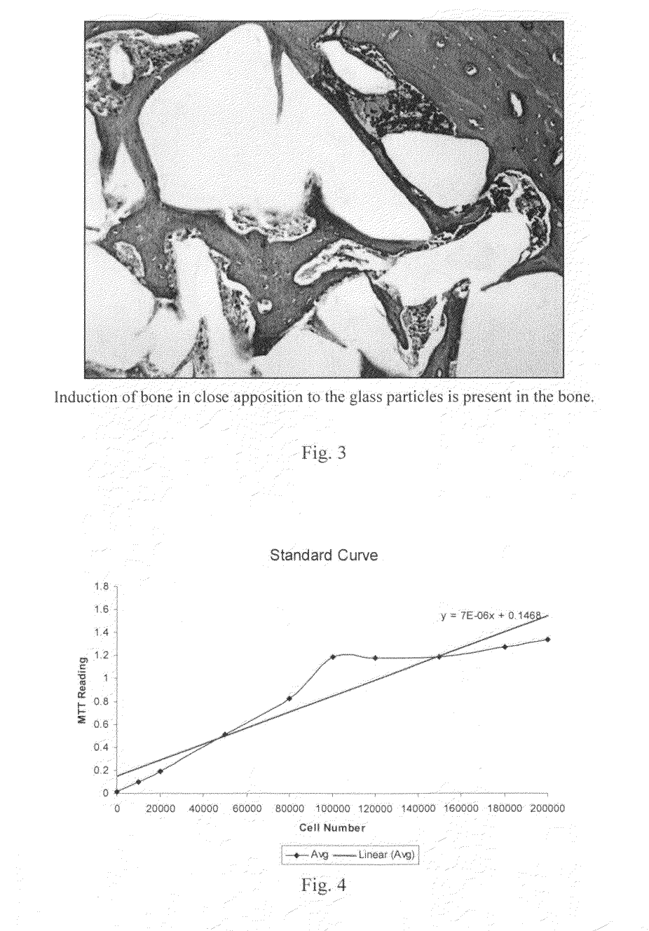

FIG. 3 is an image showing indication of bone in close opposition to glass particles present in the bone;

FIG. 4 is a plot of MTT v. cell number;

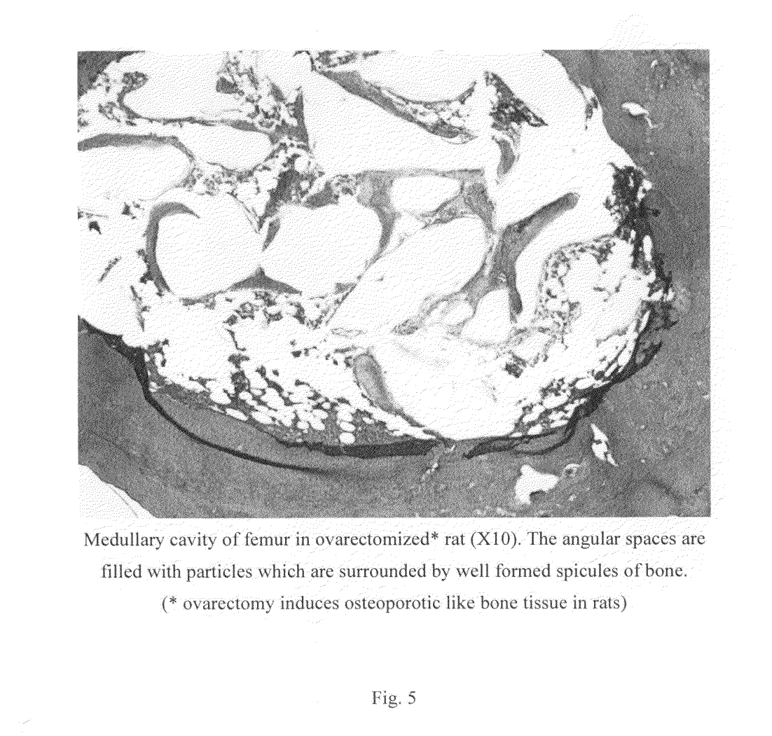

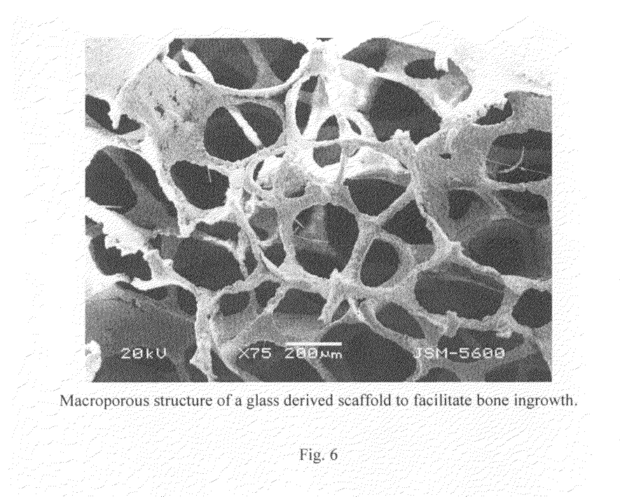

FIG. 5 is an image showing a medullary cavity of the femur of an ovarectomized rat; and

FIG. 6 is an image showing macroporous structure of a glass-derived scaffold to facilitate bone ingrowth.

### GRAFT MATERIALS

The invention provides a range of glasses based on Ca—Sr—Zn—Si—Na for use as grafts. Examples are set out in Table 1 below.

TABLE 1
Glass compositions (mole fraction).

| Glass Designation | SiO₂ | ZnO | CaO | SrO | NaO |
|---|---|---|---|---|---|
| BT107 | 0.40 | 0.32 | 0.28 | 0 | 0 |
| BT 108 | 0.40 | 0.32 | 0.14 | 0.14 | 0 |
| BT 109 | 0.40 | 0.32 | 0 | 0.28 | 0 |
| BT110 | 0.40 | 0.20 | 0.10 | 0.20 | 0.10 |
| BT111 | 0.40 | 0.10 | 0.10 | 0.20 | 0.20 |
| BT112 | 0.40 | 0 | 0.10 | 0.20 | 0.30 |
| BT113 | 0.40 | 0.20 | 0 | 0.30 | 0.10 |
| BT114 | 0.40 | 0.10 | 0 | 0.30 | 0.20 |
| BT115 | 0.40 | 0 | 0 | 0.30 | 0.30 |
| BT116 | 0.40 | 0 | 0 | 0.40 | .20 |

### Glass Synthesis

To synthesize the glasses of Table 1 appropriate amounts of analytical grade silica, zinc oxide, strontium carbonate and calcium carbonate, were weighed out and mixed thoroughly in a closed plastic container. The mixtures were then packed into platinum crucibles for firing at 1480° C. (1 hr). The glass melts were then shock quenched in water.

Resulting frit was dried in an oven at 100° C. for 2 days, then ground and sieved to obtain two glass powders with varying particle size distributions; <25 μm glass powder (cell culture) and 90 to 350 μm glass powder (animal trial).

### Applications and Benefits

The primary application of the glass is as synthetic bone (including dental) graft. The glass promotes cellular metabolism, and upon implantation in living bone tissue induces bone growth at their surface. The graft is in particulate form, not in a cement. Because the glasses are synthesised with NaO there is control of the degradation rate of the graft; a feature which is advantageous in tailoring the grafts to specific patients and applications.

The main beneficial components of the glasses for graft applications are the ZnO, SrO, and NaO. The ZnO and SrO respectively degrade to provide Zn²⁺ and Sr²⁺ ions respectively. The mechanism is osteoclastic turnover. Osteoclasts attach to glass surface and release acid to degrade the glass, releasing the zinc and strontium ions. Additionally the sodium (Na) in the glass imparts water solubility, allowing glasses to degrade to their ionic components. The ions released by the glass provide:
anti-bacterial effects;
improved bone formation in place of diseased tissue;
inhibition of bone resorption; and
radiopacity.

The NaO influences the rate of ion release, and its concentration is chosen to provide a desired glass degradation rate.

The particle size may be any suitable size, but for many applications is preferably in the range of 350 to 950 μm.

The glasses exhibit beneficial effects, for example, for patients suffering from osteoporosis, namely slow degradation of glass in vivo. The degradation process releases both Zn and Sr ions into the surrounding tissue and has therapeutic effects on osteoporotic bone. Both ions (Sr²⁺ and Zn²⁺) have been shown to promote the regeneration of healthy bone in patients suffering from metabolic bone diseases like osteoporosis. These glass grafts facilitate the regeneration of healthy bone in place of diseased tissue when implanted in vivo, and prevent infection as a result of the inherent antibacterial nature of both Sr²⁺ and Zn²⁺.

There is excellent synergy between the SrO, ZnO, and NaO. The Sr²⁺ ions have better bone formation effects than the Zn²⁺ ions, but an anti-bacterial effect which is not as good. Choice of relative proportions of ZnO and SrO combined with the choice of NaO concentration to set the resorption rate allow optimisation.

In more detail Na controls the degradation rates of the grafts under physiological conditions such that the release of strontium and zinc can be controlled. By incrementally increasing the amount of Na in the glass networks, degradation properties of the grafts can be controlled, i.e. ion release profiles (Sr²⁺ and Zn²⁺) can be tailored to specific applications or patient requirements.

Both Sr and Zn impart a synergy to optimise the bone grafts as both elements provide, to differing extents, antibacterial and biological properties on the bone grafts. Zn plays a major role in wound healing, and prevention of infection at the implant site, while playing a lesser role in influencing bone metabolism in favour of the formation of healthy bone, and diminishing the loss of healthy bone. Concurrently and synergistically, Sr plays a major role in influencing the formation of healthy bone, by improving bone formation and limiting bone resorption, while playing a lesser role in limiting infection at the implant site. The content of Na will facilitate control over the in vivo degradation rates thus allowing the grafts to be tailored to multiple applications and patient requirements. Together both elements (Zn and Sr) impart radiopaque properties on the bone grafts to facilitate roentgenographic follow-up by clinicians, or implantation of grafts under fluoroscopically guided procedures.

Treatment of osteoporotic bone with Sr containing compounds has been reported for over 40 years. However, the therapeutic effect of Sr has been neglected due to confusion in relation to radioactive isotopes of strontium. As a result of the perception that Sr is not beneficial the element has been overlooked for inclusion in glass bone grafts.

### USE EXAMPLE 1

Referring to Table 1 a surgeon may elect to use the graft designated BT107 for a patient with generally healthy bone stock who is not suffering from the effects of osteoporosis but requires a bone graft. The surgeon may elect to do so based on the fact that the influence of Sr on the patient's bone is not the overwhelming feature required for a successful procedure, rather limiting infection and controlling degradation of the graft being very important. The composition BT107 provides a loading of Zn sufficient to be antibacterial and to limit infection, whilst to a lesser extent encouraging the development of healthy bone stock. Also, the composition contains no Na, thus maximising resorption time of the graft.

### USE EXAMPLE 2

A surgeon may elect to use the graft designated BT109 for a patient suffering from the effects of osteoporosis who requires a bone graft. The surgeon may elect to do so based on the fact that the influence of Sr on the patient's bone is the most important factor for a successful procedure, whilst requiring the synergistic effect of Zn to limit infection. In this example, slow degradation of the graft is also important. The composition BT109 provides a loading of Zn sufficient to be antibacterial and to limit infection, whilst concurrently containing an equal proportion of Sr to encourage the development of healthy bone stock. The composition contains no Na, thus maximising resorption time of the graft.

### USE EXAMPLE 3

A surgeon may elect to use the graft designated BT112 for a patient suffering from the effects of osteoporosis and who requires a bone graft. The surgeon may elect to do so based on the fact that the influence of Sr on the patient's bone is a critical feature required for a successful procedure. However, in this instance infection is less of a concern and faster resorption rates are preferred. The composition BT112 provides no loading of Zn because infection control is not the overriding factor for the surgeon or the patient. However, whilst Sr will encourage the development of healthy bone stock, the surgeon is assured of its ability (in a lesser role to Zn) to limit infection.

The following are other examples of use of some of the graft materials of the invention, such as those set out in Table 1.

### Spine Surgery.

Vertebrectomies (filling material/implant), fusion both interbody and postero-laterally. Graft BT 110 would be a good choice of graft for this application.

### Cranio-Maxillofacial.

Reconstructing mandibular cyst defects and voids after tooth socket extraction augmentation of the maxillary sinus or alveolar ridge.

### As an Additive

As an additive in toothpastes to promote polishing of tooth and re-hardening of enamel, in bone cements, gels or other medical devices to encourage healthy bone formation and/or limit infection.

### Trauma and Orthopaedics.

Filling voids caused by benign tumours, cysts and osteotomies, filling defects arising from impacted fractures, refilling of cancellous bone harvesting sites, arthrodesis, non-unions and Pseudoarthrosis.

### Calculation of Network Connectivity

The network connectivity of each glass network was determined from the molar composition using Equation 1.

$$NC = \frac{No . BOs - No . NBOs}{Total No . Bridging species} \qquad Equation 1$$

Where:
BO=Bridging Oxygens
NBO=Non-Bridging Oxygens

The preferred structure of the glass graft is Q₂ or less, having network connectivity (as calculated by Equation 1) of 2 or less. This does not preclude forming successful grafts with a higher network connectivity or Q structure, rather these are preferences. All glasses described in Table 1 have network connectivity equal to 1.

### Tests

NOVABONE™ (NovaBone Products, LLC, Alachua, USA), batch #0403C1, was sourced from Musculoskeletal Transplant Foundation (Edison, USA) and used as a control for the experiments. Release of Sr and Zn ions in vivo has therapeutic effects on diseased bone and results in the generation of healthy bone in place of diseased tissue. Glasses BT107-116 of Table 1 were compared to NOVABONE™ (Control) using an ISO-approved cell culture assay.

Twenty four immature female Wistar rats aged 4-6 weeks were used for this study and were housed in groups under standard laboratory conditions. Samples of each material were implanted into the right femur under aseptic conditions under the remit of UK Home Office Project License number 40/2795.

The animals were anaesthetised using Isoflurane in oxygen; the right femur was exposed using sharp and blunt dissection and a defect created in the mid-shaft using a number 5 round stainless steel dental bur kept cool with sterile saline. Prior to implantation the test materials were washed in 70% alcohol and then washed 3 times in phosphate buffered saline; moistening the materials made them easier to manipulate; granules of a test or standard material were placed into the bone defect using fine tweezers or a dental excavator, material was gently packed into the defect prior to closing the wound with resorbable sutures. Animals were allowed to recover and kept in standard laboratory conditions for 4 weeks prior to sacrificing using a schedule one method. The right femur of each animal was dissected free and placed in formalin. The femurs were demineralised and cut into blocks prior to processing to paraffin embedded sections stained with haematoxylin and eosin.

The results of the assay (FIGS. 1 and 2) clearly show that the glasses BT107 and BT108 outperform the commercial control in almost all instances. In particular BT109, the high Sr glass, was shown to improve cellular metabolism (at 5 days) to 100% metabolic activity, as compared to NOVABONE™ which facilitated 65% metabolic activity (FIG. 2).

The glasses express almost no cytotoxic response from L929 fibroblast cells which would render them inadequate for use in vivo. Indeed it is evident that certain compositions of the grafts facilitate increased metabolic activity in L929 cells over novabone.

Upon examination of the sections of bone removed from the animals it was clear that the glass grafts induced bone growth in apposition to implanted granules (FIG. 3), indicating their potential to promote healthy bone growth.

The results from this animal trial indicate that grafts are well tolerated by bone. Furthermore the grafts facilitate bone growth and regeneration in vivo. The glasses facilitate increased metabolic activity in L929 cells over NOVABONE™.

Bone growth has also been demonstrated. As a result of the composition and improved cellular metabolism for cells in apposition to the grafts in vivo the grafts have been shown to bond directly to bone in vivo and also increase bone formation in apposition to the implanted granules. 1 mm² surface area of each glass (n=3) were exposed to 10 ml of tissue culture water, and incubated at 37° C. for each of the time periods listed below. Extracts were taken from each glass solution at each time point:
1 Day
5 Day
30 Day

6 well plates were seeded with L929 mouse fibroblasts at a specific cell density and left to adhere for 6 hours. At this time the L929 fibroblasts were exposed to 100 μl aliquots of extractant. This extractant was taken from each of the four samples at the various time points previously mentioned and was repeated three times for each specific extractant. The samples were left for 24 hrs at which point the MTT assay was employed to assess the effect of the extracts on the cellular metabolic activity.

Having exposed the L929 fibroblasts to the various extractants for 24 hrs, the culture media was removed and replaced by 500 μl/well of medium. The MTT assay is then added in an amount equal to 10% of the culture medium volume/well. The cultures were then re-incubated at 37° C. for 2 hours. After the incubation period, the cultures were removed from the incubator and the resultant formazan crystals were dissolved by adding an amount of MTT solubilization solution (10% Triton x-100 in Acidic Isopropanol. (0.1 n HCI)) equal to the original culture medium volume. Once the crystals were fully dissolved, the absorbance was measured at a wavelength of 570 nm.

Controls used for this investigation were L929 fibroblasts exposed to 100 μl aliquots of biological water. Again these extracts were taken at three time points. These cells were assumed to have metabolic activities of 100% and the percentage metabolic activity of the L929 fibroblast cells exposed to the various extracts of the test solutions was calculated relative to this.

L929 fibroblast cells were seeded at various known densities and left to adhere for 6 hours under normal conditions, not allowing for any cell doubling to arise. The metabolic activity of these cells was assessed resulting in a metabolic activity standard curve at specific cell densities. This was repeated three times and the average results are shown here.

FIG. 4 shows an MTT reading vs. cell number plot. FIG. 5 shows medullary cavity of femur in ovarectomized rat, showing angular spaces filled with particles which are surrounded by well-formed spicules of bone. It is to be noted that ovarectomy induces osteoporotic-like bone tissue in rats.

The invention is not limited to the embodiments described but may be varied in construction and detail within the scope of the claims. For example, the graft may not be provided in granular form. It may be sintered or cast into a desired shape. This may, for example, allow for a load-bearing application. FIG. 6 shows macroporous structure of a glass-derived scaffold to facilitate bone ingrowth, where the glass was sintered into a shape for a load-bearing application.

1. A synthetic bone graft glass having a composition including SrO and ZnO.

2. A synthetic bone graft glass as claimed in claim 1, wherein the glass comprises SiO₂, ZnO, CaO, and SrO.

3. A synthetic bone graft glass as claimed in claim 1, wherein the glass further comprises NaO.

4. A synthetic bone graft glass as claimed in claim 1, wherein the concentration of ZnO is in the range of 0.05 to 0.50 mole fraction.

5. A synthetic bone graft material as claimed in claim 4, wherein the concentration of ZnO is in the range of 0.10 to 0.32 mole fraction.

6. A synthetic bone graft glass as claimed in claim 1, wherein the concentration of SrO is in the range of 0.05 to 0.50 mole fraction.

7. A synthetic bone graft glass as claimed in claim 6, wherein the SrO concentration is in the range of 0.14 to 0.40 mole fraction.

8. A synthetic bone graft glass as claimed in claim 3, wherein the concentration of NaO is in the range of 0.05 to 0.5 mole fraction.

9. A synthetic bone graft glass as claimed in claim 3, wherein the NaO concentration is in the range of 0.1 to 0.3 mole fraction

10. A synthetic bone graft glass as claimed in claim 1, wherein the glass structure is Q₂ or less, having network connectivity of 2 or less.

11. A synthetic bone graft glass as claimed in claim 1, wherein the glass further comprises NaO; and wherein the glass structure is Q₂ or less, having network connectivity of 2 or less.

12. A synthetic bone graft glass as claimed in claim 10, wherein the network connectivity is 1.

13. A synthetic bone graft glass as claimed in claim 1, wherein the glass is in particulate form.

14. A synthetic bone graft glass as claimed in claim 1, wherein the glass further comprises NaO; and wherein the glass is in particulate form.

15. A synthetic bone graft glass as claimed in claim 13, wherein the particle size is in the range of 350 μm to 950 μm.

16. A synthetic bone graft glass as claimed in any of claim 1, wherein the glass is in the form of a load-bearing body.

17. A synthetic bone graft glass as claimed in any of claim 1, wherein the glass further comprises NaO; and wherein the glass is in the form of a load-bearing body

18. A synthetic bone graft comprising a glass as claimed in claim 1.

19. A synthetic bone graft comprising a glass as claimed in claim 1; and wherein the glass further comprises NaO.

20. A graft as claimed in claim 18 wherein the graft is a void-filling graft.

21. A graft as claimed in claim 18, wherein the graft is a cranio-maxillo facial graft.

22. A toothpaste comprising a glass as claimed in claim 1.

23. A toothpaste comprising a glass as claimed in claim 1; and wherein the glass further comprises NaO.

24. A method of synthesising a glass of claim 1, in which SrO and ZnO concentrations are chosen to optimise target bone formation and anti-bacterial activities, the SrO component providing better bone formation properties and the ZnO component providing better anti-bacterial properties.

25. A method as claimed in claim 24, comprising the step of including NaO at a proportion chosen according to desired rate of release of Sr and Zn ions.

26. A method as claimed in claim 24, comprising the further step of forming the glass into a load-bearing body.
